(19) United States
(12) Patent Application Publication
(10) Pub. No.: US 2023/0399354 A1
(43) Pub. Date: Dec. 14, 2023

(54) PREPARATION METHOD OF 2'-SUBSTITUTED PYRIMIDINE NUCLEOSIDE

(71) Applicant: BEIJING RIBIO PHARMA CO., LTD., Beijing (CN)

(72) Inventors: Yazhou LI, Beijing (CN); Yibiao LIU, Beijing (CN); Guanshen ZHOU, Beijing (CN); Zhenchang CHEN, Beijing (CN); Hongjuan ZHANG, Beijing (CN)

(73) Assignee: BEIJING RIBIO PHARMA CO., LTD., Beijing (CN)

(21) Appl. No.: 17/999,080

(22) PCT Filed: Sep. 16, 2022
(86) PCT No.: PCT/CN2022/119170

§ 371 Date: Nov. 17, 2022

(30) Foreign Application Priority Data
Jan. 21, 2022 (CN) 202210072500.0

(51) Int. Cl. C07H 19/067

(52) U.S. Cl. CPC C07H 19/067

(57) ABSTRACT

The present disclosure provides a preparation method of a 2′-substituted pyrimidine nucleoside, including the following steps: subjecting a compound of Formula I or Formula II to a series of process including dehydration, selective 5′-protection, ring-opening reaction using magnesium alkoxide and deprotection to obtain a 2′-substituted pyrimidine nucleoside of Formula VII or Formula VIII. In the present disclosure, the preparation method has desirable universality for different substrates; the ring-opening with a protected anhydrous pyrimidine nucleoside improves a solubility of the substrate, with milder reaction conditions than those of a traditional synthetic route; the formation of a dimer is avoided during the ring-opening to improve the yield; in addition, an intermediate (5′-O-bis-p-methoxytrityl-protected 2′-substituted pyrimidine nucleoside) can be directly used in synthesis of a corresponding phosphoramidite monomer, with a wider potential for use.

### CROSS REFERENCE TO RELATED APPLICATION

The present application claims the priority to Chinese Patent Application No. CN202210072500.0, titled “PREPARATION METHOD OF 2′-SUBSTITUTED PYRIMIDINE NUCLEOSIDE”, filed with China National Intellectual Property Administration (CNIPA) on Jan. 21, 2022, which is incorporated herein by reference in its entirety.

### TECHNICAL FIELD

The present disclosure relates to a preparation method of a 2′-substituted pyrimidine nucleoside, and belongs to the field of chemical synthesis.

### BACKGROUND

Nucleic acid drugs refer to nucleic acids or compounds closely related to the nucleic acids that can be used to treat diseases, including natural nucleotides and chemically-modified nucleotides. The nucleic acid drug specifically recognizes an endogenous nucleic acid sequence through a base complementary pairing mechanism, thereby exerting a therapeutic effect. In addition to gene therapy, nucleic acids used for therapy can also inhibit the expression of abnormal proteins associated with diseases by inhibiting the expression of DNAs or RNAs, without affecting the expression of other proteins. Compared with antibody drugs, the nucleic acid drugs show more excellent efficacy and safety than the antibody drugs, and are conducive to mass production by pharmaceutical companies due to a relatively small molecular weight. These characteristics make nucleic acid drugs promising for previously difficult-to-treat cancers and genetic diseases, as well as diseases caused by viral infections such as influenza.

In recent years, nucleic acid drugs have made great progresses. However, nucleic acids are unstable in human body, are easily degraded by nucleases after entering the blood, and are easily cleared by the kidneys, with a short half-life; moreover, exogenous nucleic acid molecules are immunogenic and can easily cause immune responses in human body. Chemical modification provides a good solution to the above problems. The modification of 2′-hydroxyl in a ribose structure is an extremely common technique, and nucleoside monomers with structurally modified at 2′-hydroxyl widely exist in the nucleic acid drugs that are currently on the market and under development. Therefore, there is an increasingly growing market demand for such nucleoside monomers.

At present, the most straightforward preparation for 2′-hydroxyl-substituted pyrimidine nucleosides was developed by Saroj K. Roy and Jin-yan Tang. The dehydrated uridine or dehydrated cytidine was reacted with magnesium alkoxide resulting in direct ring-opening to obtain a compound in which hydrogen of the 2′-hydroxyl was substituted by alkyl (Roy S K, Tang J. Efficient Large Scale Synthesis of 2′-O-Alkyl Pyrimidine Ribonucleosides [J]. Org. Process. Res. Dev., 2000, 4, 170-171). The yields of 2′-O-methyluridine and 2′-O-methylcytidine from the dehydrated uridine and the dehydrated cytidine were 89% and 76%, respectively. However, this method has harsh reaction conditions; moreover, during the ring-opening, 5% to 10% of dimers may be formed; furthermore, after quenching followed by the ring-opening, magnesium salts are difficult to be removed, such that a resulting product is prone to being unqualified due to residues on ignition (>5%). In addition, a preparation method was developed by Urtzi Legorburu, Colin B. Reese, and Quanlai Song. The dehydrated uridine or dehydrated cytidine was reacted with aluminum methoxyethoxide resulting in direct ring-opening to obtain a compound in which hydrogen of the 2′-hydroxyl was substituted by methoxyethyl (Legorburu U, Reese C B, Song Q. Conversion of uridine into 2′-O-(2-methoxyethyl) uridine and 2′-O-(2-methoxyethyl) cytidine [J]. Tetrahedron, 1999, 55, 5635-5640). The method also has the above problems. Due to the above difficulties and defects, these methods are difficult to achieve industrialization and large-scale production.

Therefore, it is of great significance to develop a synthesis process of 2′-substituted pyrimidine nucleosides that is easy to be industrialized.

### SUMMARY

A purpose of the present disclosure is to provide a preparation method of a 2′-substituted pyrimidine nucleoside. The method can stably obtain qualified products and is easy to be industrialized.

The present disclosure provides a preparation method of a 2′-substituted pyrimidine nucleoside, including the following steps:
1) subjecting a compound of Formula I or Formula II to dehydration to obtain a compound of Formula III; where

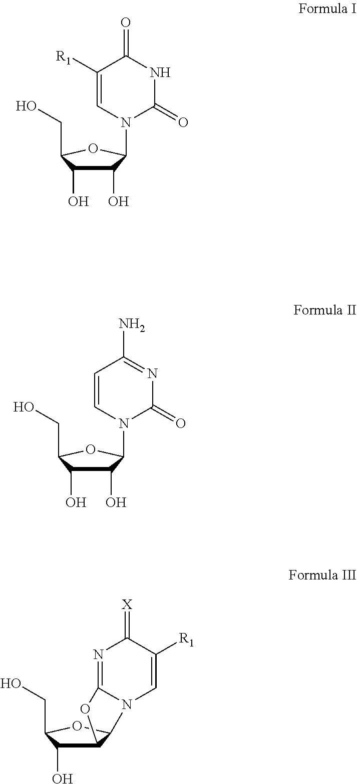

in formulas I and III, R₁ is selected from the group consisting of hydrogen and methyl, and X is selected from the group consisting of ═O and ═NH;
2) subjecting the compound of Formula III to selective 5′-protection to obtain a compound of Formula IV; where

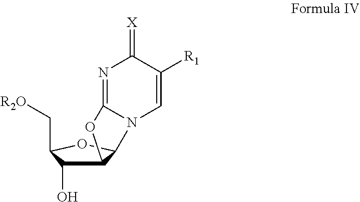

in Formula IV, R₂ is bis-p-methoxytrityl, and X is selected from the group consisting of ═O and ═NH;
3) under the action of magnesium alkoxide, subjecting the compound of Formula IV to ring-opening to obtain a compound of Formula V or Formula VI; where

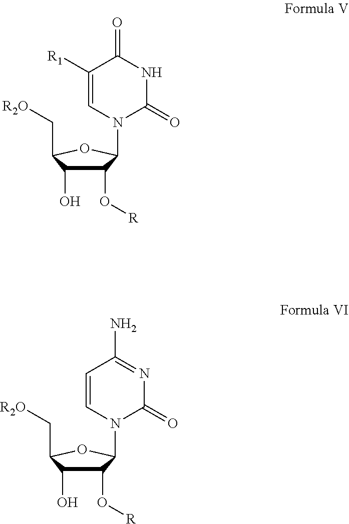

in Formula V and Formula VI, R₁ has a same definition as R₁ in Formula I, R₂ has a same definition as R₂ in Formula IV, and R is selected from the group consisting of methyl and methoxyethyl; and
4) subjecting the compound of Formula V or Formula VI to deprotection to obtain a 2′-substituted pyrimidine nucleoside of Formula VII or Formula VIII; where

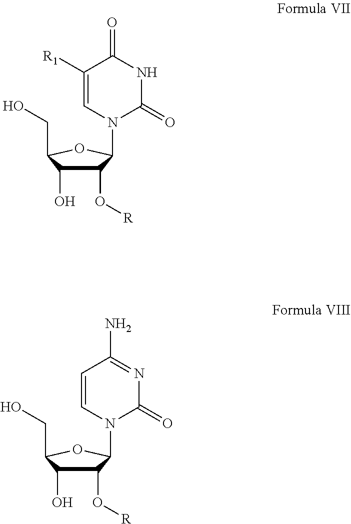

in Formula VII and Formula VIII, R has a same definition as R in Formula V and Formula VI, and R₁ has a same definition as R₁ in Formula I.

In the preparation method, in step 1), the dehydration is conducted in the presence of diphenyl carbonate and an alkali;
the alkali is selected from the group consisting of NaHCO₃, NaOH, and NaOCH₃; and
the dehydration is conducted in N,N-dimethylformamide.

In the preparation method, the dehydration is conducted by:
a reaction at 80° C. for 4 h to 12 h to reflux; and
the compound of Formula I or the compound of Formula II, the diphenyl carbonate, and the alkali have a molar ratio of 1:(1.1-1.5):(0.02-0.05).

In the preparation method, in step 1), after the dehydration is completed, the preparation method further includes: precipitating a product by slowly reducing to room temperature, conducting filtration, reslurrying an obtained filter cake with methanol or dichloromethane-methanol, and conducting re-filtration to obtain the compound of Formula III;

the reslurrying is conducted with the methanol when using the compound of Formula I as a material; and

the reslurrying is conducted with the dichloromethane-methanol when using the compound of Formula II as the material.

In the preparation method, in step 2), the selective 5′-protection is conducted under the action of a protective reagent, pyridine, and 4-dimethylaminopyridine;
the protective reagent is bis-p-methoxytriphenylchloromethane; and
the selective 5′-protection is conducted in dichloromethane or 1,2-dichloroethane.

In the preparation method, the selective 5′-protection is conducted by:
a reaction at 20° C. to 80° C. for 4 h to 24 h; and
the compound of Formula III, the protective reagent, the pyridine, and the 4-dimethylaminopyridine have a molar ratio of 1:(1.05-1.2):(1.05-1.2):(0.02-0.05).

In the preparation method, in step 2), after the selective 5′-protection is completed, the preparation method further includes: layering by water; washing an obtained organic phase with water, a saturated sodium bicarbonate solution, and a saturated saline solution in sequence, conducting evaporation to dryness, and subjecting an obtained residue to recrystallization with n-hexane-dichloromethane, n-heptane-dichloromethane, or n-heptane-ethyl acetate to obtain the compound of Formula IV;
when in Formula III, R₁ is hydrogen, and X is ═O, the recrystallization is conducted with the n-hexane-dichloromethane;
when in Formula III, R₁ is methyl, and X is ═O, the recrystallization is conducted with the n-heptane-dichloromethane; and
when in Formula III, X is ═NH, the recrystallization is conducted with the n-heptane-ethyl acetate.

In the preparation method, in step 3), a preparation method of the magnesium alkoxide includes:
soaking a magnesium rod or a magnesium sheet in dilute hydrochloric acid, rinsing with an alcohol, suspending in the alcohol after wipe-drying, and heating until the magnesium rod or the magnesium sheet is completely dissolved;
the alcohol is selected from the group consisting of methanol and ethylene glycol monomethyl ether; and
the ring-opening is conducted in the alcohol serving as a solvent.

In the preparation method, the ring-opening is conducted by:
a reaction at 60° C. to 150° C. for 5 h to 24 h; and
the compound of Formula IV and the magnesium alkoxide have a molar ratio of 1:(2-8).

In the preparation method, in step 3), after the ring-opening is completed, the preparation method further includes: neutralizing excessive magnesium methoxide or magnesium methoxyethoxide with acetic acid, filtering, conducting evaporation on an obtained filtrate to dryness; dispersing an obtained residue in dichloromethane, washing with water three times, subjecting an obtained organic phase to concentration, and conducting recrystallization with n-hexane-dichloromethane, n-hexane-ethyl acetate, n-heptane-dichloromethane, or n-heptane-ethyl acetate to obtain the compound of Formula V or Formula VI;
when in Formula III, R₁ is hydrogen, X is ═O, and the alcohol is methanol, the excessive magnesium methoxide is neutralized, and the recrystallization is conducted with the n-hexane-dichloromethane;
when in Formula III, R₁ is hydrogen, X is ═O, and the alcohol is ethylene glycol monomethyl ether, the excessive magnesium methoxyethoxide is neutralized, and the recrystallization is conducted with the n-hexane-ethyl acetate;
when in Formula III, R₁ is methyl, X is ═O, and the alcohol is methanol, the excessive magnesium methoxide is neutralized, and the recrystallization is conducted with the n-hexane-dichloromethane;
when in Formula III, R₁ is methyl, X is ═O, and the alcohol is ethylene glycol monomethyl ether, the excessive magnesium methoxyethoxide is neutralized, and the recrystallization is conducted with the n-heptane-dichloromethane;
when in Formula III, X is ═NH, and the alcohol is ethylene glycol monomethyl ether, the excessive magnesium methoxyethoxide is neutralized, and the recrystallization is conducted with the n-hexane-dichloromethane; and

when in Formula III, X is ═NH, and the alcohol is methanol, the excessive magnesium methoxide is neutralized, and the recrystallization is conducted with the n-heptane-ethyl acetate.

In the preparation method, in step 4), the deprotection is conducted in an acid or a mixed solution of the acid and water;
the acid is formic acid, or acetic acid containing 0 to 3 halogen substituents; and
in the mixed solution of the acid and water, the acid and water have a volume ratio of 1:(0-1).

In the preparation method, the deprotection is conducted by:
a reaction at 20° C. to 80° C. for 2 h to 16 h.

In the preparation method, in step 4), after the deprotection is completed, the preparation method further includes: conducting vacuum concentration, adding 200 mL of water, conducting extraction three times with dichloromethane, and discarding resulting organic phase; subjecting an obtained aqueous phase to evaporation under reduced pressure to dryness, and conducting recrystallization on a resulting residue with ethanol-ethyl acetate.

In the preparation method of the present disclosure, a dosage of the solvent in each step is the dosage known to those skilled in the art.

Compared with the prior art, the present disclosure has the following advantages:
In the present disclosure, the preparation method has desirable universality for different substrates;
the ring-opening with a protected anhydrous pyrimidine nucleoside improves a solubility of the substrate, with milder reaction conditions than those of a traditional synthetic route;
the formation of a dimer is avoided during the ring-opening to improve the yield;
salts can be stably and effectively removed, such that an obtained product has no residual inorganic salt with a stable quality; and
an intermediate (5′-O-bis-p-methoxytrityl-protected 2′-substituted pyrimidine nucleoside) can be directly used in synthesis of a corresponding phosphoramidite monomer, with a wider potential for use.

### DETAILED DESCRIPTION OF THE EMBODIMENTS

The present disclosure is further described below with reference to the accompanying drawings and examples.

The materials, reagents, and the like used in the following examples are all commercially available, unless otherwise specified.

In the following examples, an HPLC purity is detected with reference to General Principle 0512 of “Chinese Pharmacopoeia (2020 Edition)” (Volume IV), and a residue on ignition is detected with reference to General Principle 0841 of “Chinese Pharmacopoeia (2020 Edition)” (Volume IV).

### Example 1 Synthesis of 2′-O-methyluridine

A reaction formula was as follows:

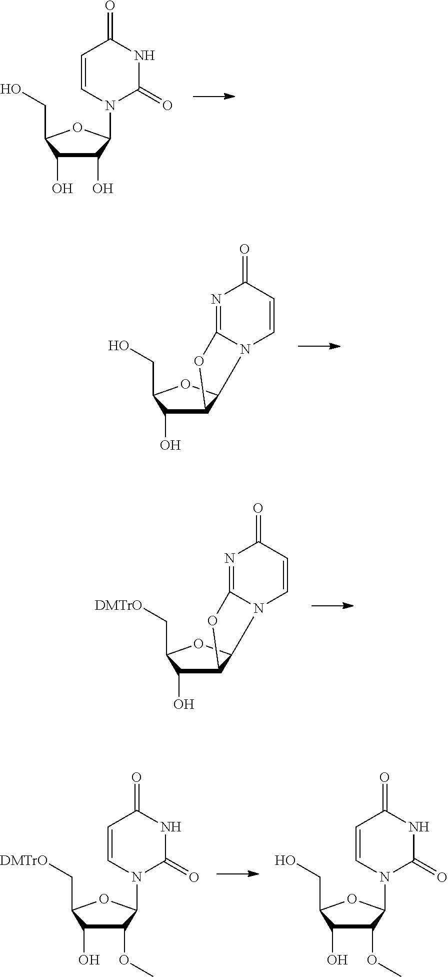

### Step 1: Synthesis of 2,2′-O-cyclouridine

50.0 g (205 mmol) of uridine was dissolved in 200 mL of N,N-dimethylformamide, 48.3 g (225 mmol) of diphenyl carbonate and 0.34 g (4.09 mmol) of sodium bicarbonate were added, and a reaction was conducted by heating at 120° C. for 8 h, followed by monitoring until an end of the reaction; a product was precipitated by slowly cooling to a room temperature, subjected to filtration, an obtained filter cake was reslurried with methanol, and subjected to filtration to obtain 44.1 g of the 2,2′-O-cyclouridine as a white powder with a yield of 95.2%.

### Step 2: Synthesis of 5′-O-bis-p-methoxytrityl-2,2′-O-cyclouridine

40.0 g (177 mmol) of the 2,2′-O-cyclouridine was dissolved in 200 mL of dichloromethane, 62.9 g (186 mmol) of bis-p-methoxytriphenylchloromethane, 14.7 g (186 mmol) of pyridine, and g (3.54 mmol) of 4-dimethylaminopyridine were added, and a reaction was conducted by heating at 20° C. for 24 h, followed by monitoring until an end of the reaction; layering was conducted with water; an obtained organic phase was washed with water, a saturated sodium bicarbonate solution, and a saturated saline solution in sequence, evaporated to dryness, an obtained residue was recrystallized with n-hexane-dichloromethane to obtain 87.8 g of the as a white waxy solid with a yield of 93.9%.

### Step 3: Synthesis of 5′-O-bis-p-methoxytrityl-2′-O-methyluridine

22.1 g (908 mmol) of a magnesium rod was soaked in dilute hydrochloric acid, quickly rinsed with anhydrous methanol and wipe-dried, added to 400 mL of anhydrous methanol, and heated slightly to initiate a reaction; after the magnesium rod was completely dissolved, 80.0 g (151 mmol) of the 5′-O-bis-p-methoxytrityl-2,2′-O-cyclouridine was added, and a reaction was conducted by heating at 120° C. for 12 h, followed by monitoring until an end of the reaction; acetic acid was added to neutralize excessive magnesium methoxide, filtration was conducted, an obtained filtrate was evaporated to dryness, an obtained residue was dispersed in dichloromethane and washed with water 3 times; an obtained organic phase was concentrated, and recrystallized with n-hexane-dichloromethane to obtain 81.6 g of the 5′-O-bis-p-methoxytrityl-2′-O-methyluridine as a white waxy solid with a yield of 96.2%.

### Step 4: Synthesis of 2′-O-methyluridine

80.0 g (143 mmol) of the 5′-O-bis-p-methoxytrityl-2′-O-methyluridine was dissolved in a mixture including 300 mL of acetic acid and 100 mL of water, and a reaction was conducted by heating at 80° C. for 8 h, followed by monitoring until an end of the reaction; a resulting product was subjected to vacuum concentration, added with 200 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 33.9 g of the 2′-O-methyluridine as an off-white powder with a yield of 92.0%, an HPLC purity of greater than 98%, and a residue on ignition of 0.05%.

Structural characterization data was as follows: ¹H NMR (400 MHz, DMSO-d₆) δ 11.34 (br s, 1H), 7.93 (d, J=8.0 Hz, 1H), 5.85 (d, J=5.0 Hz, 1H), 5.65 (d, J=8.0 Hz, 1H), 5.19 (br s, 1H), 5.16 (br s, 1H), 4.12 (t, J=4.8, 1H), 3.85 (dt, J=4.6, 3.2 Hz, 1H), 3.78 (t, J=5.0 Hz, 1H), 3.64 (dd, J=12.2, 3.2 Hz, 1H), 3.56 (dd, J=12.2, 3.2 Hz, 1H), 3.35 (s, 3H).

### Example 2 Synthesis of 2′-O-methylcytidine

A reaction formula was as follows:

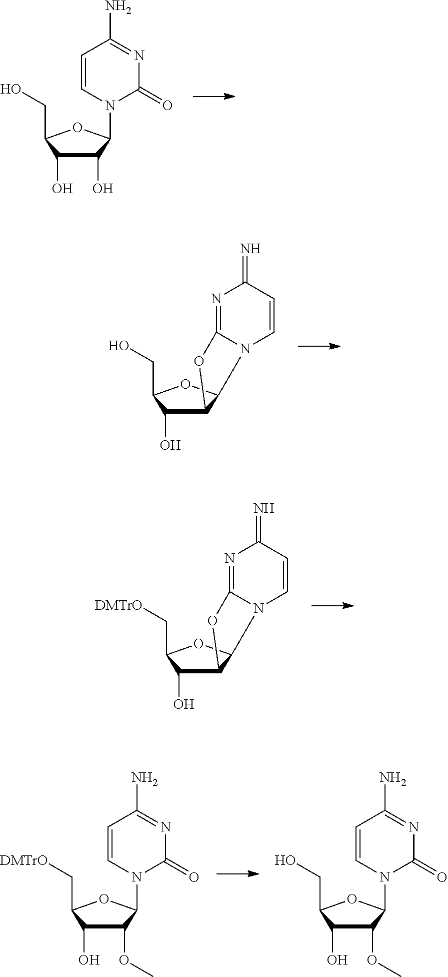

### Step 1: Synthesis of 2,2′-anhydrocytidine

50.0 g (206 mmol) of cytidine was dissolved in 200 mL of N,N-dimethylformamide, 52.9 g (247 mmol) of diphenyl carbonate and 0.25 g (6.17 mmol) of sodium hydroxide were added, and a reaction was conducted by heating at 80° C. for 12 h, followed by monitoring until an end of the reaction; a product was precipitated by slowly cooling to a room temperature, subjected to filtration, an obtained filter cake was reslurried with dichloromethane-methanol, and subjected to filtration to obtain 42.3 g of the 2,2′-anhydrocytidine as a white powder with a yield of 91.4%.

### Step 2: Synthesis of 5′-O-bis-p-methoxytrityl-2,2′-anhydrocytidine

40.0 g (178 mmol) of the 2,2′-anhydrocytidine was dissolved in 200 mL of 1,2-dichloroethane, 72.2 g (213 mmol) of bis-p-methoxytriphenylchloromethane, 16.9 g (213 mmol) of pyridine, and 0.43 g (3.55 mmol) of 4-dimethylaminopyridine were added, and a reaction was conducted by heating at 60° C. for 12 h, followed by monitoring until an end of the reaction; layering was conducted with water; an obtained organic phase was washed with water, a saturated sodium bicarbonate solution, and a saturated saline solution in sequence, evaporated to dryness, an obtained residue was recrystallized with n-heptane-ethyl acetate to obtain 83.9 g of the 5′-O-bis-p-methoxytrityl-2,2′-anhydrocytidine as a white solid with a yield of 89.5%.

### Step 3: Synthesis of 5′-O-bis-p-methoxytrityl-2′-O-methylcytidine

29.5 g (1.21 mol) of a magnesium rod was soaked in dilute hydrochloric acid, quickly rinsed with anhydrous methanol and wipe-dried, added to 400 mL of anhydrous methanol, and heated slightly to initiate a reaction; after the magnesium rod was completely dissolved, 80.0 g (152 mmol) of the 5′-O-bis-p-methoxytrityl-2,2′-anhydrocytidine was added, and a reaction was conducted by heating at 60° C. for 24 h, followed by monitoring until an end of the reaction; acetic acid was added to neutralize excessive magnesium methoxide, filtration was conducted, an obtained filtrate was evaporated to dryness, an obtained residue was dispersed in dichloromethane and washed with water 3 times; an obtained organic phase was concentrated, and recrystallized with n-heptane-ethyl acetate to obtain 68.8 g of the 5′-O-bis-p-methoxytrityl-2′-O-methylcytidine as a white solid with a yield of 81.1%.

### Step 4: Synthesis of 2′-O-methylcytidine

60 g (107 mmol) of the 5′-O-bis-p-methoxytrityl-2′-O-methylcytidine was dissolved in 300 mL of acetic acid, and a reaction was conducted by heating at 40° C. for 8 h, followed by monitoring until an end of the reaction; a resulting product was subjected to vacuum concentration, added with 600 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 24.6 g of the 2′-O-methylcytidine as an off-white powder with a yield of 89.2%, an HPLC purity of greater than 98%, and a residue on ignition of 0.04%.

Structural characterization data was as follows: ¹H NMR (400 MHz, DMSO-d₆) δ 7.90 (d, J=7.4 Hz, 1H), 7.24 (br s, 1H), 7.19 (br s, 1H), 5.84 (d, J=4.0 Hz, 1H), 5.72 (d, J=7.4 Hz, 1H), 5.10 (t, J=5.2 Hz, 1H), 5.05 (d, J=6.2 Hz, 1H), 4.05 (q, J=5.6 Hz, 1H), 3.81 (dt, J=6.0, 3.0 Hz, 1H), 3.63-3.69 (m, 2H), 3.52-3.58 (m, 1H), 3.82 (s, 3H).

### Example 3 Synthesis of 2′-O-methyl-5-methyluridine

A reaction formula was as follows:

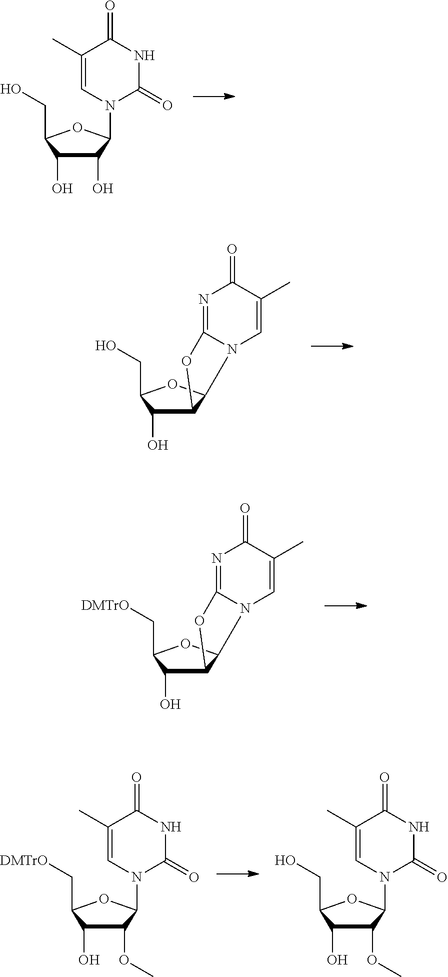

### Step 1: Synthesis of 2,2′-anhydro-5-methyluridine

50.0 g (194 mmol) of 5-methyluridine was dissolved in 200 mL of N,N-dimethylformamide, 62.2 g (290 mmol) of diphenyl carbonate and 0.52 g (9.68 mmol) of sodium methoxide were added, and a reaction was conducted by heating for 4 h, followed by monitoring until an end of the reaction; a product was precipitated by slowly cooling to a room temperature, subjected to filtration, an obtained filter cake was reslurried with methanol, and subjected to filtration to obtain 43.7 g of the 2,2′-anhydro-5-methyluridine as a white powder with a yield of 94.0%.

### Step 2: Synthesis of 5′-O-bis-p-methoxytrityl-2,2′-anhydro-5-methyluridine

40.0 g (167 mmol) of the 2,2′-anhydro-5-methyluridine was dissolved in 200 mL of 1,2-dichloroethane, 59.2 g (175 mmol) of bis-p-methoxytriphenylchloromethane, 13.8 g (175 mmol) of pyridine, and 0.41 g (3.33 mmol) of 4-dimethylaminopyridine were added, and a reaction was conducted by heating at 80° C. for 4 h, followed by monitoring until an end of the reaction; layering was conducted with water; an obtained organic phase was washed with water, a saturated sodium bicarbonate solution, and a saturated saline solution in sequence, evaporated to dryness, an obtained residue was recrystallized with n-heptane-dichloromethane to obtain 82.6 g of the as a white waxy solid with a yield of 91.4%.

### Step 3: Synthesis of 5′-O-bis-p-methoxytrityl-2′-O-methyl-5-methyluridine

21.5 g (885 mmol) of a magnesium rod was soaked in dilute hydrochloric acid, quickly rinsed with anhydrous methanol and wipe-dried, added to 400 mL of anhydrous methanol, and heated slightly to initiate a reaction; after the magnesium rod was completely dissolved, 80.0 g (147 mmol) of the 5′-O-bis-p-methoxytrityl-2,2′-anhydro-5-methyluridine was added, and a reaction was conducted by heating at 150° C. for 5 h, followed by cooling to a room temperature; acetic acid was added to neutralize excessive magnesium methoxide, filtration was conducted, an obtained filtrate was evaporated to dryness, an obtained residue was dispersed in dichloromethane and washed with water 3 times; an obtained organic phase was concentrated, and recrystallized with n-hexane-dichloromethane to obtain 79.8 g of the as a white waxy solid with a yield of 94.2%.

### Step 4: Synthesis of 2′-O-methyl-5-methyluridine

70.0 g (122 mmol) of the 5′-O-bis-p-methoxytrityl-2′-O-methyl-5-methyluridine was dissolved in a mixture including 140 mL of trifluoroacetic acid and 140 mL of water, and a reaction was conducted by heating at 40° C. for 2 h, followed by monitoring until an end of the reaction; a resulting product was subjected to vacuum concentration, added with 150 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 31.1 g of the 2′-O-methyl-5-methyluridine as an off-white powder with a yield of 93.8%, an HPLC purity of greater than 98%, and a residue on ignition of Structural characterization data was as follows: ¹H NMR (400 MHz, DMSO-d₆) δ 11.23 (br s, 1H), 7.78 (s, 1H), 5.84 (d, J=5.6 Hz, 1H), 5.14 (t, J=5.2 Hz, 1H), 5.10 (d, J=6.2 Hz, 1H), 4.12-4.16 (m, 1H), 3.82-3.85 (m, 1H), 3.77-3.81 (m, 1H), 3.55-3.69 (m, 2H), 3.35 (s, 3H), 1.77 (s, 3H).

### Example 4 Synthesis of 2′-O-methoxyethyluridine

A reaction formula was as follows:

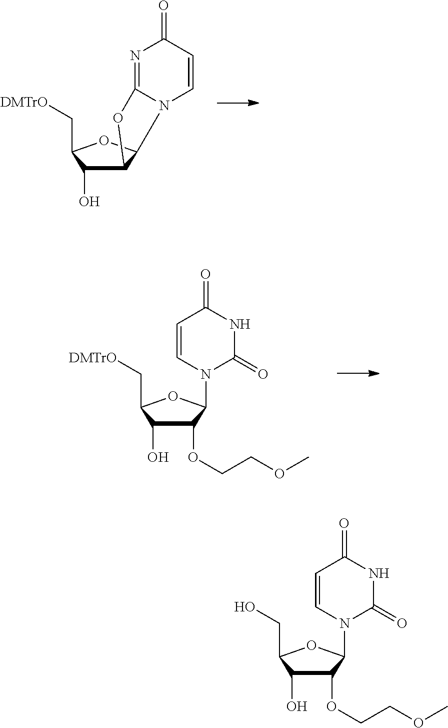

Step 1 and Step 2 were the same as Step 1 and Step 2 of Example 1, so as to obtain 5′-O-bis-p-methoxytrityl-2,2′-O-cyclouridine.

### Step 3: Synthesis of 5′-O-bis-p-methoxytrityl-2′-O-methoxyethyluridine

11.0 g (454 mmol) of a magnesium rod was soaked in dilute hydrochloric acid, quickly rinsed with ethylene glycol monomethyl ether and wipe-dried, added to 400 mL of ethylene glycol monomethyl ether, and heated to initiate a reaction; after the magnesium rod was completely dissolved, 80.0 g (151 mmol) of the 5′-O-bis-p-methoxytrityl-2,2′-O-cyclouridine was added, and a reaction was conducted by heating at 90° C. for 12 h, followed by monitoring until an end of the reaction; acetic acid was added to neutralize excessive magnesium methoxyethoxide, filtration was conducted, an obtained filtrate was evaporated to dryness, an obtained residue was dispersed in dichloromethane and washed with water 3 times; an obtained organic phase was concentrated, and recrystallized with n-hexane-ethyl acetate to obtain 81.2 g of the 5′-O-bis-p-methoxytrityl-2,2′-O-methoxyethyluridine as a white waxy solid with a yield of 88.7%.

### Step 4: Synthesis of 2′-O-methoxyethyluridine

60.0 g (143 mmol) of the 5′-O-bis-p-methoxytrityl-2′-O-methoxyethyluridine and 200 g of chloroacetic acid were dissolved in 100 mL of water, and a reaction was conducted by heating at for 2 h, followed by monitoring until an end of the reaction; a resulting product was added with 500 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 26.8 g of the 2′-O-methoxyethyluridine as an off-white powder with a yield of 89.4%, an HPLC purity of greater than 98%, and a residue on ignition of 0.05%.

Structural characterization data was as follows: ¹H NMR (400 MHz, DMSO-d₆) δ 11.33 (br s, 1H), 7.93 (d, J=8.2 Hz, 1H), 5.84 (d, J=5.0 Hz, 1H), 5.65 (d, J=8.2 Hz, 1H), 4.10 (t, J=4.6 Hz, 1H), 3.95 (t, J=5.0 Hz, 1H), 3.84-3.87 (m, 1H), 3.61-3.71 (m, 3H), 3.55 (dd, J=12.2, 3.2 Hz, 1H), 3.44 (t, J=4.8 Hz, 2H), 3.22 (s, 3H).

### Example 5 Synthesis of 2′-O-methoxyethylcytidine

A reaction formula was as follows:

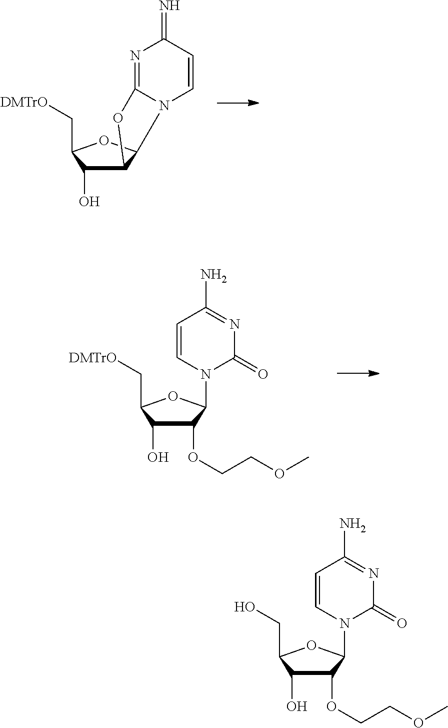

Step 1 and Step 2 were the same as Step 1 and Step 2 of Example 2, so as to obtain 5′-O-bis-p-methoxytrityl-2,2′-anhydrocytidine.

### Step 3: Synthesis of 5′-O-bis-p-methoxytrityl-2′-O-methoxyethylcytidine

11.1 g (455 mmol) of a magnesium rod was soaked in dilute hydrochloric acid, quickly rinsed with ethylene glycol monomethyl ether and wipe-dried, added to 400 mL of ethylene glycol monomethyl ether, and heated to initiate a reaction; after the magnesium rod was completely dissolved, 80.0 g (152 mmol) of the 5′-O-bis-p-methoxytrityl-2,2′-anhydrocytidine was added, and a reaction was conducted by heating at 90° C. for 12 h, followed by monitoring until an end of the reaction; acetic acid was added to neutralize excessive magnesium methoxyethoxide, filtration was conducted, an obtained filtrate was evaporated to dryness, an obtained residue was dispersed in dichloromethane and washed with water 3 times; an obtained organic phase was concentrated, and recrystallized with n-hexane-dichloromethane to obtain 73.5 g of the 5′-O-bis-p-methoxytrityl-2′-O-methoxyethylcytidine as a white solid with a yield of 80.3%.

### Step 4: Synthesis of 2′-O-methoxyethylcytidine

60 g (107 mmol) of the 5′-O-bis-p-methoxytrityl-2′-O-methoxyethylcytidine was dissolved in a mixture including 225 mL of acetic acid and 75 mL of water, and a reaction was conducted by heating at 60° C. for 8 h, followed by monitoring until an end of the reaction; a resulting product was subjected to vacuum concentration, added with 300 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 22.6 g of the 2′-O-methoxyethylcytidine as an off-white powder with a yield of 75.5%, an HPLC purity of greater than 98%, and a residue on ignition of 0.05%.

Structural characterization data was as follows: ¹H NMR (400 MHz, DMSO-d₆) δ 7.90 (d, J=7.6 Hz, 1H), 7.21 (br s, 1H), 7.17 (br s, 1H), 5.83 (d, J=4.0 Hz, 1H), 5.72 (d, J=7.6 Hz, 1H), 4.04 (t, J=5.6 Hz, 1H), 3.79-3.84 (m, 2H), 3.65-3.76 (m, 3H), 3.55 (dd, J=12.2, 3.2 Hz, 1H), 3.46 (t, J=4.8 Hz, 2H), 3.23 (s, 3H).

### Example 6 Synthesis of 2′-O-methoxyethyl-5-methyluridine

A reaction formula was as follows:

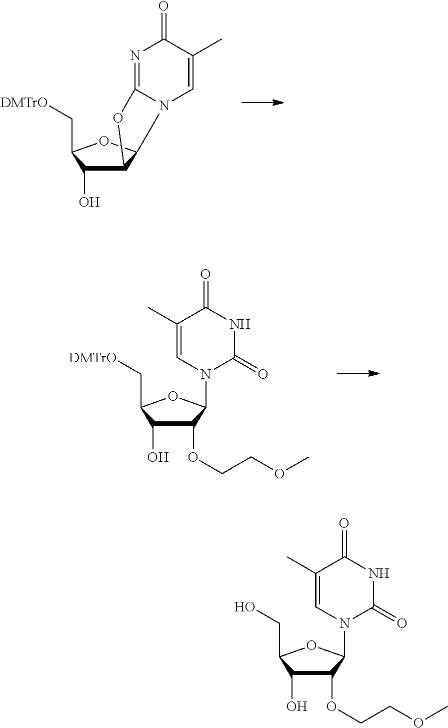

Step 1 and Step 2 were the same as Step 1 and Step 2 of Example 3, so as to obtain 5′-O-bis-p-methoxytrityl-2,2′-anhydro-5-methyluridine.

### Step 3: Synthesis of 5′-O-bis-p-methoxytrityl-2′-O-methoxyethyl-5-methyluridine

7.17 g (295 mmol) of a magnesium rod was soaked in dilute hydrochloric acid, quickly rinsed with ethylene glycol monomethyl ether and wipe-dried, added to 400 mL of ethylene glycol monomethyl ether, and heated to initiate a reaction; after the magnesium rod was completely dissolved, 80.0 g (147 mmol) of the 5′-O-bis-p-methoxytrityl-2,2′-anhydro-5-methyluridine was added, and a reaction was conducted by heating at 120° C. for 5 h, followed by cooling to a room temperature; acetic acid was added to neutralize excessive magnesium methoxyethoxide, filtration was conducted, an obtained filtrate was evaporated to dryness, an obtained residue was dispersed in dichloromethane and washed with water 3 times; an obtained organic phase was concentrated, and recrystallized with n-heptane-dichloromethane to obtain 82.8 g of the 5′-O-bis-p-methoxytrityl-2′-O-methoxyethyl-5-methyluridine as a white waxy solid with a yield of 90.8%.

### Step 4: Synthesis of 2′-O-methoxyethyl-5-methyluridine

80.0 g (129 mmol) of the 5′-O-bis-p-methoxytrityl-2′-O-methoxyethyl-5-methyluridine was dissolved in a mixture including 160 mL of trifluoroacetic acid and 160 mL of water, and a reaction was conducted by heating at 40° C. for 2 h, followed by monitoring until an end of the reaction; a resulting product was subjected to vacuum concentration, added with 240 mL of water, extracted 3 times with dichloromethane, and an obtained organic phase was discarded; an obtained aqueous phase was evaporated under reduced pressure to dryness, and an obtained residue was recrystallized with ethanol-ethyl acetate to obtain 36.2 g of the 2′-O-methoxyethyl-5-methyluridine as an off-white powder with a yield of 88.5%, an HPLC purity of greater than 98%, and a residue on ignition of 0.02%.

Structural characterization data was as follows: ¹H NMR (400 MHz, DMSO-d₆) δ 11.30 (br s, 1H), 7.79 (s, 1H), 5.85 (d, J=5.0 Hz, 1H), 4.10 (t, J=4.6 Hz, 1H), 3.95 (t, J=5.0 Hz, 1H), 3.83-3.87 (m, 1H), 3.60-3.71 (m, 3H), 3.55 (dd, J=12.2, 3.2 Hz, 1H), 3.44 (t, J=4.8 Hz, 2H), 3.22 (s, 3H), 1.78 (s, 3H).

The above description of examples is merely provided to help illustrate the method of the present disclosure and a core idea thereof. It should be noted that several improvements and modifications may be made by persons of ordinary skill in the art without departing from the principle of the present disclosure, and these improvements and modifications should also fall within the protection scope of the present disclosure. Various amendments to these embodiments are apparent to those of professional skill in the art, and the general principles defined herein may be implemented in other embodiments without departing from the spirit or scope of the present disclosure. Thus, the present disclosure is not limited to the examples shown herein but falls within the widest scope consistent with the principles and novel features disclosed herein.

1-13. (canceled)

14. A preparation method of a 2′-substituted pyrimidine nucleoside, comprising the following steps:
1) subjecting a compound of Formula I or Formula II to dehydration to obtain a compound of Formula III; wherein 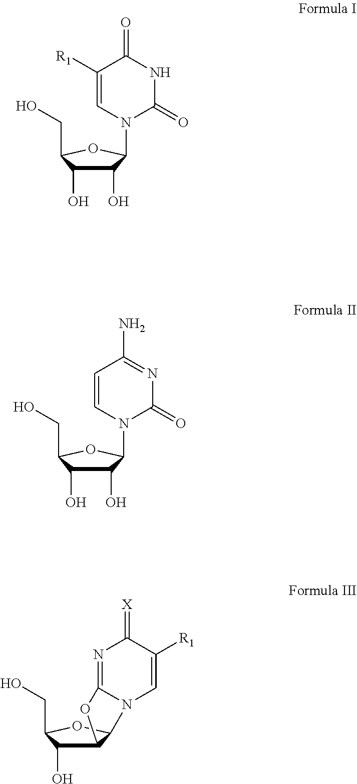
in formulas I and III, R₁ is selected from the group consisting of hydrogen and methyl, and X is selected from the group consisting of ═O and ═NH;
2) subjecting the compound of Formula III to selective 5′-protection to obtain a compound of Formula IV; wherein 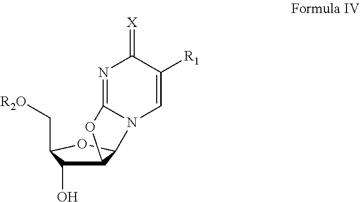
in Formula IV, R₂ is bis-p-methoxytrityl, and X is selected from the group consisting of ═O and ═NH;
3) under the action of magnesium alkoxide, subjecting the compound of Formula IV to ring-opening to obtain a compound of Formula V or Formula VI; wherein 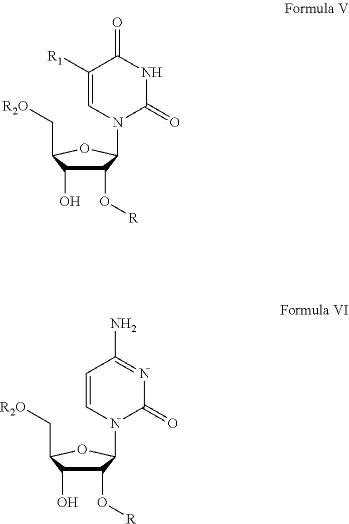
in Formula V and Formula VI, R₁ has a same definition as R₁ in Formula I, R₂ has a same definition as R₂ in Formula IV, and R is selected from the group consisting of methyl and methoxyethyl; and
4) subjecting the compound of Formula V or Formula VI to deprotection to obtain a 2′-substituted pyrimidine nucleoside of Formula VII or Formula VIII; wherein 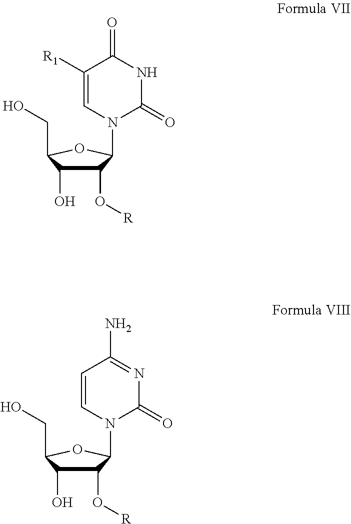
in Formula VII and Formula VIII, R has a same definition as R in Formula V or Formula VI, and R₁ has a same definition as R₁ in Formula I.

15. The preparation method according to claim 14, wherein in step 1), the dehydration is conducted in the presence of diphenyl carbonate and an alkali;
the alkali is selected from the group consisting of NaHCO₃, NaOH, and NaOCH₃; and
the dehydration is conducted in N,N-dimethylformamide.

16. The preparation method according to claim 15, wherein the dehydration is conducted by:
a reaction at 80° C. for 4 h to 12 h to reflux; and
the compound of Formula I or the compound of Formula II, the diphenyl carbonate, and the alkali have a molar ratio of 1:(1.1-1.5):(0.02-0.05).

17. The preparation method according to claim 14, wherein in step 1), after the dehydration is completed, the preparation method further comprises: precipitating a product by slowly reducing to room temperature, conducting filtration, reslurrying an obtained filter cake with methanol or dichloromethane-methanol, and conducting filtration to obtain the compound of Formula III;
the reslurrying is conducted with the methanol when using the compound of Formula I as a material; and
the reslurrying is conducted with the dichloromethane-methanol when using the compound of Formula II as the material.

18. The preparation method according to claim 15, wherein in step 1), after the dehydration is completed, the preparation method further comprises: precipitating a product by slowly reducing to a room temperature, conducting filtration, reslurrying an obtained filter cake with methanol or dichloromethane-methanol, and conducting filtration to obtain the compound of Formula III;
the reslurrying is conducted with the methanol when using the compound of Formula I as a material; and
the reslurrying is conducted with the dichloromethane-methanol when using the compound of Formula II as the material.

19. The preparation method according to claim 16, wherein in step 1), after the dehydration is completed, the preparation method further comprises: precipitating a product by slowly reducing to a room temperature, conducting filtration, reslurrying an obtained filter cake with methanol or dichloromethane-methanol, and conducting filtration to obtain the compound of Formula III;
the reslurrying is conducted with the methanol when using the compound of Formula I as a material; and
the reslurrying is conducted with the dichloromethane-methanol when using the compound of Formula II as the material.

20. The preparation method according to claim 14, wherein in step 2), the selective 5′-protection is conducted under the action of a protective reagent, pyridine, and 4-dimethylaminopyridine;
the protective reagent is bis-p-methoxytriphenylchloromethane; and
the selective 5′-protection is conducted in dichloromethane or 1,2-dichloroethane.

21. The preparation method according to claim 20, wherein the selective 5′-protection is conducted by:
a reaction at 20° C. to 80° C. for 4 h to 24 h; and
the compound of Formula III, the protective reagent, the pyridine, and the 4-dimethylaminopyridine have a molar ratio of 1:(1.05-1.2):(1.05-1.2):(0.02-0.05).

22. The preparation method according to claim 14, wherein in step 2), after the selective 5′-protection is completed, the preparation method further comprises: layering by water; washing an obtained organic phase with water, a saturated sodium bicarbonate solution, and a saturated saline solution in sequence, conducting evaporation to dryness, and subjecting an obtained residue to recrystallization with n-hexane-dichloromethane, n-heptane-dichloromethane, or n-heptane-ethyl acetate to obtain the compound of Formula IV;
when in Formula III, R₁ is hydrogen, and X is ═O, the recrystallization is conducted with the n-hexane-dichloromethane;
when in Formula III, R₁ is methyl, and X is ═O, the recrystallization is conducted with the n-heptane-dichloromethane; and
when in Formula III, X is ═NH, the recrystallization is conducted with the n-heptane-ethyl acetate.

23. The preparation method according to claim 20, wherein in step 2), after the selective is completed, the preparation method further comprises: layering by water; washing an obtained organic phase with water, a saturated sodium bicarbonate solution, and a saturated saline solution in sequence, conducting evaporation to dryness, and subjecting an obtained residue to recrystallization with n-hexane-dichloromethane, n-heptane-dichloromethane, or n-heptane-ethyl acetate to obtain the compound of Formula IV;
when in Formula III, R₁ is hydrogen, and X is ═O, the recrystallization is conducted with the n-hexane-dichloromethane;
when in Formula III, R₁ is methyl, and X is ═O, the recrystallization is conducted with the n-heptane-dichloromethane; and
when in Formula III, X is ═NH, the recrystallization is conducted with the n-heptane-ethyl acetate.

24. The preparation method according to claim 21, wherein in step 2), after the selective is completed, the preparation method further comprises: layering by water; washing an obtained organic phase with water, a saturated sodium bicarbonate solution, and a saturated saline solution in sequence, conducting evaporation to dryness, and subjecting an obtained residue to recrystallization with n-hexane-dichloromethane, n-heptane-dichloromethane, or n-heptane-ethyl acetate to obtain the compound of Formula IV;
when in Formula III, R₁ is hydrogen, and X is ═O, the recrystallization is conducted with the n-hexane-dichloromethane;
when in Formula III, R₁ is methyl, and X is ═O, the recrystallization is conducted with the n-heptane-dichloromethane; and
when in Formula III, X is ═NH, the recrystallization is conducted with the n-heptane-ethyl acetate.

25. The preparation method according to claim 14, wherein in step 3), a preparation method of the magnesium alkoxide comprises:
soaking a magnesium rod or a magnesium sheet in dilute hydrochloric acid, rinsing with an alcohol, suspending in the alcohol after wipe-drying, and heating until the magnesium rod or the magnesium sheet is completely dissolved;
the alcohol is selected from the group consisting of methanol and ethylene glycol monomethyl ether; and
the ring-opening is conducted in the alcohol serving as a solvent.

26. The preparation method according to claim 22, wherein the ring-opening is conducted by:
a reaction at 60° C. to 150° C. for 5 h to 24 h; and
the compound of Formula IV and the magnesium alkoxide have a molar ratio of 1:(2-8).

27. The preparation method according to claim 14, wherein in step 3), after the ring-opening is completed, the preparation method further comprises: neutralizing excessive magnesium methoxide or magnesium methoxyethoxide with acetic acid, filtering, conducting evaporation on an obtained filtrate to dryness; dispersing an obtained residue in dichloromethane, washing with water three times, subjecting an obtained organic phase to concentration, and conducting recrystallization with n-hexane-dichloromethane, n-hexane-ethyl acetate, n-heptane-dichloromethane, or n-heptane-ethyl acetate to obtain the compound of Formula V or Formula VI;
when in Formula III, R₁ is hydrogen, X is ═O, and the alcohol is methanol, the excessive magnesium methoxide is neutralized, and the recrystallization is conducted with the n-hexane-dichloromethane;
when in Formula III, R₁ is hydrogen, X is ═O, and the alcohol is ethylene glycol monomethyl ether, the excessive magnesium methoxyethoxide is neutralized, and the recrystallization is conducted with the n-hexane-ethyl acetate;
when in Formula III, R₁ is methyl, X is ═O, and the alcohol is methanol, the excessive magnesium methoxide is neutralized, and the recrystallization is conducted with the n-hexane-dichloromethane;
when in Formula III, R₁ is methyl, X is ═O, and the alcohol is ethylene glycol monomethyl ether, the excessive magnesium methoxyethoxide is neutralized, and the recrystallization is conducted with the n-heptane-dichloromethane;
when in Formula III, X is ═NH, and the alcohol is ethylene glycol monomethyl ether, the excessive magnesium methoxyethoxide is neutralized, and the recrystallization is conducted with the n-hexane-dichloromethane; and
when in Formula III, X is ═NH, and the alcohol is methanol, the excessive magnesium methoxide is neutralized, and the recrystallization is conducted with the n-heptane-ethyl acetate.

28. The preparation method according to claim 25, wherein in step 3), after the ring-opening is completed, the preparation method further comprises: neutralizing excessive magnesium methoxide or magnesium methoxyethoxide with acetic acid, filtering, conducting evaporation on an obtained filtrate to dryness; dispersing an obtained residue in dichloromethane, washing with water three times, subjecting an obtained organic phase to concentration, and conducting recrystallization with n-hexane-dichloromethane, n-hexane-ethyl acetate, n-heptane-dichloromethane, or n-heptane-ethyl acetate to obtain the compound of Formula V or Formula VI;
when in Formula III, R₁ is hydrogen, X is ═O, and the alcohol is methanol, the excessive magnesium methoxide is neutralized, and the recrystallization is conducted with the n-hexane-dichloromethane;
when in Formula III, R₁ is hydrogen, X is ═O, and the alcohol is ethylene glycol monomethyl ether, the excessive magnesium methoxyethoxide is neutralized, and the recrystallization is conducted with the n-hexane-ethyl acetate;
when in Formula III, R₁ is methyl, X is ═O, and the alcohol is methanol, the excessive magnesium methoxide is neutralized, and the recrystallization is conducted with the n-hexane-dichloromethane;
when in Formula III, R₁ is methyl, X is ═O, and the alcohol is ethylene glycol monomethyl ether, the excessive magnesium methoxyethoxide is neutralized, and the recrystallization is conducted with the n-heptane-dichloromethane;
when in Formula III, X is ═NH, and the alcohol is ethylene glycol monomethyl ether, the excessive magnesium methoxyethoxide is neutralized, and the recrystallization is conducted with the n-hexane-dichloromethane; and
when in Formula III, X is ═NH, and the alcohol is methanol, the excessive magnesium methoxide is neutralized, and the recrystallization is conducted with the n-heptane-ethyl acetate.

29. The preparation method according to claim 26, wherein in step 3), after the ring-opening is completed, the preparation method further comprises: neutralizing excessive magnesium methoxide or magnesium methoxyethoxide with acetic acid, filtering, conducting evaporation on an obtained filtrate to dryness; dispersing an obtained residue in dichloromethane, washing with water three times, subjecting an obtained organic phase to concentration, and conducting recrystallization with n-hexane-dichloromethane, n-hexane-ethyl acetate, n-heptane-dichloromethane, or n-heptane-ethyl acetate to obtain the compound of Formula V or Formula VI;
when in Formula III, R₁ is hydrogen, X is ═O, and the alcohol is methanol, the excessive magnesium methoxide is neutralized, and the recrystallization is conducted with the n-hexane-dichloromethane;
when in Formula III, R₁ is hydrogen, X is ═O, and the alcohol is ethylene glycol monomethyl ether, the excessive magnesium methoxyethoxide is neutralized, and the recrystallization is conducted with the n-hexane-ethyl acetate;
when in Formula III, R₁ is methyl, X is ═O, and the alcohol is methanol, the excessive magnesium methoxide is neutralized, and the recrystallization is conducted with the n-hexane-dichloromethane;
when in Formula III, R₁ is methyl, X is ═O, and the alcohol is ethylene glycol monomethyl ether, the excessive magnesium methoxyethoxide is neutralized, and the recrystallization is conducted with the n-heptane-dichloromethane;
when in Formula III, X is ═NH, and the alcohol is ethylene glycol monomethyl ether, the excessive magnesium methoxyethoxide is neutralized, and the recrystallization is conducted with the n-hexane-dichloromethane; and
when in Formula III, X is ═NH, and the alcohol is methanol, the excessive magnesium methoxide is neutralized, and the recrystallization is conducted with the n-heptane-ethyl acetate.

30. The preparation method according to claim 14, wherein in step 4), the deprotection is conducted in an acid or a mixed solution of the acid and water;
the acid is formic acid, or acetic acid containing 0 to 3 halogen substituents; and
in the mixed solution of the acid and water, the acid and water have a volume ratio of 1:(0-1).

31. The preparation method according to claim 24, wherein the deprotection is conducted by:
a reaction at 20° C. to 80° C. for 2 h to 16 h.

32. The preparation method according to claim 14, wherein in step 4), after the deprotection is completed, the preparation method further comprises: conducting vacuum concentration, adding 200 mL of water, conducting extraction three times with dichloromethane, and discarding a resulting organic phase; subjecting an obtained aqueous phase to evaporation under reduced pressure to dryness, and conducting recrystallization on a resulting residue with ethanol-ethyl acetate.

33. The preparation method according to claim 30, wherein in step 4), after the deprotection is completed, the preparation method further comprises: conducting vacuum concentration, adding 200 mL of water, conducting extraction three times with dichloromethane, and discarding a resulting organic phase; subjecting an obtained aqueous phase to evaporation under reduced pressure to dryness, and conducting recrystallization on a resulting residue with ethanol-ethyl acetate.
